(19) United States
(12) Patent Application Publication
(10) Pub. No.: US 2011/0294994 A1
(43) Pub. Date: Dec. 1, 2011

(54) Gentamicin Separation Method

(75) Inventor: Jonathan Grote, Green Oaks, IL (US)

(73) Assignee: ABBOTT LABORATORIES, Abbott Park, IL (US)

(21) Appl. No.: 13/106,249

(22) Filed: May 12, 2011

Related U.S. Application Data
(60) Provisional application No. 61/348,783, filed on May 27, 2010.

(51) Int. Cl. C07H 1/06

(52) U.S. Cl. 536/13.6

(57) ABSTRACT

The invention provides more effective methods of separating the components of gentamicin using a UV active protecting group suitable for use with HPLC.

### CROSS-REFERENCE TO RELATED APPLICATIONS

This application claims priority to U.S. provisional application Ser. No. 61/348,783, filed May 27, 2010, the contents of which are hereby incorporated by reference in its entirety.

### FIELD OF THE INVENTION

This invention relates to a method of preparing gentamicin, and in particular relates to more effective methods of separating the components of gentamicin using a UV active protecting group suitable for use with HPLC.

### BACKGROUND OF THE INVENTION

Gentamicin is an aminoglycoside antibiotic complex naturally synthesized by Micromonospora, a Gram-positive genus of bacteria widely found in water and soil (Abou-Zied et al., J. Appl. Chem. & Biotech. 1976, 26, 318-22) This antibiotic is active against a wide variety of bacteria, and works by binding the 30S subunit of the bacterial ribosome, which interrupts bacterial protein synthesis (Savic, M et al., J. Bacteriology 2008, 190, 5855-61). Gentamicin is typically not administered orally, due to poor adsorption from the digestive tract, but instead is given intravenously, intramuscularly, or topically to treat bacterial infections (Mugabe, C, et al., Antimicrob. Agents Chemotherapy. 2006, 50, 2016-22). Serum concentrations of gentamicin must be carefully monitored, since overdoses can result in permanent damage to the balance and orientation components of the inner ear, as well as nephrotoxic effects in renal cells, potentially leading to renal failure. (Sundin, D. P. et al., J. Am. Soc. Nephr. 2001, 12, 114-123). Commercially manufactured by fermentation, gentamicin consists of three separate major components which differ only by the presence of methyl groups in various locations on each molecule (Chu, J.; Zhang et al., Process Biochemistry (Oxford, UK) 2002, 38(5), 815-820). The relative proportions of these components can vary widely depending on how the antibiotic was cultured or isolated, presenting challenges to the development of a conjugate for immunoassay. Selective reaction of one of the five different amino groups present in all components offers a further challenge. Traditional methods of large-scale gentamicin separation is difficult (Maehr, H. et al., J. Chrom. 1967, 30, 572; Wagman, P. et al. J. Chrom. 1968, 34, 210-17). Gentamicin has acid-sensitive functionality, limiting protection/deprotection. Further, the components of gentamicin are not UV active, which limits detectability during chromatographic separaction. New methods to separate gentamicin components on a large scale are therefore needed.

### SUMMARY OF THE INVENTION

The invention provides a method of separating a gentamicin component, comprising the steps of protecting a gentamicin component mixture with a UV active protecting group, and purifying with HPLC. In another embodiment, the present invention provides a method of separating a gentamicin component, comprising the steps of protecting a gentamicin component mixture with a UV active protecting group, and purifying with HPLC, wherein the method further comprises deprotecting the gentamicin component. In another embodiment, the present invention provides a method of separating a gentamicin component, comprising the steps of protecting a gentamicin component mixture with a UV active protecting group, and purifying with HPLC, wherein the method further comprises deprotecting the gentamicin component, wherein further the deprotected gentamicin is not significantly degraded.

In another embodiment, the present invention provides a method of separating a gentamicin component, comprising the steps of protecting a gentamicin component mixture with a UV active protecting group, and purifying with HPLC, wherein the HPLC employs an acetonitrile eluent. In another embodiment, the eluent is an isocratic acetonitrile aqueous trifluoroacetic acid eluent. In another embodiment, the eluent has less than a 65:35 ratio of acetonitrile to 0.05% aqueous trifluoroacetic acid. In another embodiment, the eluent is isocratic 63:37 acetonitrile:0.05% aqueous trifluoroacetic acid.

In another embodiment, the present invention provides a method of separating a gentamicin component, comprising the steps of protecting a gentamicin component mixture with a UV active protecting group, and purifying with HPLC, wherein the protecting group is selected from the group consisting of selected from a carbamate, an amide, an imide, benzyl, dimethoxyphenyl, dibenzosuberyl, trityl, picolyl N oxide, pyridyl N oxide, benzylidene, a benzylidene derivative, diphenylmethyl, a metal chelate, a phosphorus derivative, benzenesulfonyl, and a benzenesulfonyl derivative. In another embodiment, the protecting group is a carbamate. In another embodiment, the protecting group is a benzyl carbamate. In another embodiment, the protecting group is benzyl carbamate substituted with chloro.

In another embodiment, the present invention provides a method of separating a gentamicin component, comprising the steps of protecting a gentamicin component mixture with a UV active protecting group, and purifying with HPLC, wherein the gentamicin component is gentamicin C1a.

In another embodiment, the present invention provides a method of separating gentamicin C1a from a mixture of gentamicin components, comprising the steps of protecting a gentamicin component mixture with benzyl carbamate, and purifying with HPLC using an isocratic acetonitrile aqueous trifluoroacetic acid eluent wherein the eluent has less than a 65:35 ratio of acetonitrile to 0.05% aqueous trifluoroacetic acid.

### BRIEF DESCRIPTION OF THE DRAWINGS

FIG. 1 illustrates the chemical structure of the three different molecular forms of gentamicin (C₁, C₁ₐ and C₂).

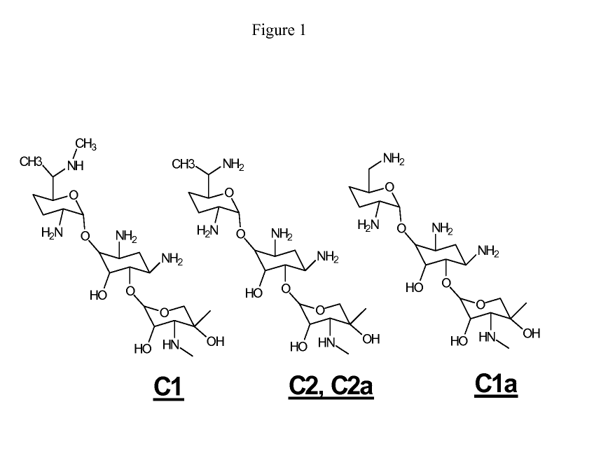

FIG. 2 illustrates HPLC of the crude reaction mixture of benzyl carbamate (cBz)-protected gentamicin (the integral of the tracer peak at 10.229 min is 23.6% at 254 nm). The first peak was identified as C1a by ESMS (deprotected material showed M+H)+ at 450.5 (other components 464.5). Isolated conjugate ESMS showed (M+H)+ 1016 with fragmentation to peaks at 840 and 695, consistent with top ring coupling. Conjugate ¹H NMR showed a dd at δ 4.23 (J=7.3/12.0 Hz), shifted from gentamicin C1a (d δ 3.78 (J=7.2 Hz, 12.0 Hz), consistent with methylene coupling.

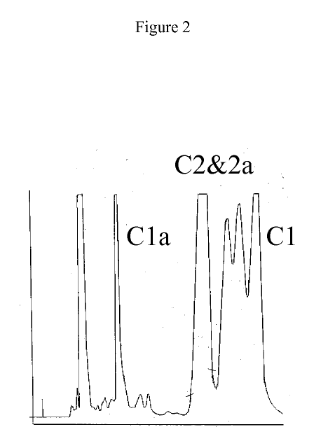

### DETAILED DESCRIPTION OF THE INVENTION

### I. Definitions

As used herein, the term “blocking group” or “protecting group” means groups which render the blocked or protected amino groups inert to subsequent desired chemical manipulation, but which can be easily removed at the end of the synthetic sequence without cleaving the desired amino group.

As used herein, the term “eluent” is used in its conventional meaning in chromatography, i.e. a solution capable of perturbing the interaction between the solid phase (adsorbent matrix) and product (gentamicin component) and promoting selective dissociation of the product from the solid phase.

As used herein, the term “gentamicin component” means any of the major or minor gentamicin components, including gentacmicins C₁, C₁ₐ, and C₂ and its two steroisomers of C₂ (C₂ and C₂ₐ).

As used herein, the term “UV active” means a compound is capable of absorbing UV light, allowing detection during HPLC.

As used herein, the term “significantly degraded” means the primary chemical bond structure of the compound is altered from what it was originally.

### I. Embodiments

The invention provides a method of for separating a gentamicin component, comprising the steps of protecting a gentamicin component mixture with a UV active protecting group, and purifying with HPLC. In one embodiment, the gentamicin component is gentomicin C1a. In another embodiment, the gentamicin component is gentamycin C1. In another embodiment, the gentamicin component is gentamycin C2.

A. Protecting Groups

In one embodiment, the present invention provides a method of for separating a gentamicin component, comprising the steps of

protecting a gentamicin component mixture with a UV active protecting group selected from a carbamate, an amide, an imide, benzyl, dimethoxyphenyl, dibenzosuberyl, trityl, picolyl N oxide, pyridyl N oxide, benzylidene, a benzylidene derivative, diphenylmethyl, a metal chelate, a phosphorus derivative, benzenesulfonyl, and a benzenesulfonyl derivative; and purifying with HPLC.

Suitable carbamates which can be used as protecting groups include fluorenylmethyl (FMOC) and its sulfo and dibromo derivatives; di-t-butyl-dioxo-tetrahydrothioxanthylmethyl (DBD-TMOC), methoxyphenacyl, phenylethyl, pyridylethyl, quinolyl, substituted benzyl groups (methoxy, dimethoxy, nitro, dimethoxynitro, bromo, chloro, dichloro), toluenesulfonylethyl, chloroacyloxybenzyl, and benzisoxazolylmethyl. Suitable amides which can be used as protecting groups include phenylacyl, phenylpropyl, picolinyl, benzoyl, phenylacyl, and phenylbenzyl. Suitable imides that can be used as protecting groups includes phthalimide, diphenylmaleimide, dimethylpyrrole, and nitropyrrone.

Other suitable protecting groups include benzyl, dimethoxyphenyl, dibenzosuberyl, trityl, picolyl N oxide, pyridyl N oxide, benzylidene and its derivatives (methosy, nitro, hydroxy, chloro hydroxy, chlorohydroxyphenyl) diphenylmethyl, metal chelates, phosphorus derivatives, benzenesulfonyl and its derivatives (methylbenzenesulfonyl=tosyl, trimethyl methoxy, dimethylmethoxy, pentamethyl, tetramethylmethoxy, trimethyl, dimethoxymethyl), anthracenesulfonyl, benzylsulfonyl, and phenacyl.

In one embodiment, the protecting group is a carbamate. In another embodiment, the protecting group is a benzyl carbamate. In another embodiment, the protecting group is benzyl carbamate substituted with chloro. In another embodiment, the benzyl carbamate is substituted with a single chloro.

B. Eluents

In one embodiment, the present invention provides a method of separating a gentamicin component, comprising the steps of protecting a gentamicin component mixture with a UV active protecting group, and purifying with HPLC, wherein the HPLC employs an acetonitrile eluent. In one embodiment, the eluent is methanol in a methanol aqueous acetonitrile mixture. In another embodiment, the eluent is an isocratic acetonitrile aqueous trifluoroacetic acid eluent. In another embodiment, the eluent has less than a 70:30 ratio of acetonitrile to 0.05% aqueous trifluoroacetic acid. In another embodiment, the eluent has less than a 65:35 ratio of acetonitrile to 0.05% aqueous trifluoroacetic acid. In another embodiment, the eluent is isocratic 63:37 acetonitrile:0.05% aqueous trifluoroacetic acid.

C. Solid Phases for Preparative High Pressure Liquid Chromatography (Prep HPLC) Separation of Gentamicin

In one embodiment, the present invention provides a method of separating a gentamicin component, comprising the steps of protecting a gentamicin component mixture with a UV active protecting group, and purifying with HPLC, wherein the HPLC employs a reverse phase column.

In one embodiment, the reverse phase column is selected from μBondapak™ (Waters Lab, Milford, Mass.), DeltaPak™ C-18 (Waters Lab, Milford, Mass.), Nova-pak® C-18, Symmetry®Shield C8 and Symmetry®Shield C18 (Waters Lab, Milford, Mass.), Symmetry®300 (Waters Lab, Milford, Mass.), XTerra® (Waters Lab, Milford, Mass.), Sphereisorb® (Waters Lab, Milford, Mass.), Sunfire™ (Waters Lab, Milford, Mass.), YMC™ (Waters Lab, Milford, Mass.), including YMC™ ODS-AQ (Waters Lab, Milford, Mass.), Luna® (Phenomenex, Torrence, Calif.) and Hypersil® (ThermoFisher Scientific, Waltham, Mass.). In one embodiment, the reverse phase column is YMC™ ODS-AQ (Waters Lab, Milford, Mass.).

### III. Examples

The amines for all gentamicin components were protected using 6.5 equivalents of benzyl chloroformate. The protected C1a component was separated by preparative HPLC on a 47×300 cm YMC ODS AQ column, eluting isocratically with a 70:20:10 acetonitrile:water:0.05% aqueous trifluoroacetic acid gradient, detecting at 215 nm. Lyophilization provided the fully protected C1a component as a white solid. Deprotection of the C1a amine was achieved using catalytic hydrogenation. Pure Gentamicin C1a component was isolated by catalyst filtration and solvent removal. The C1a component was identified by mass spectroscopy and 1H NMR.

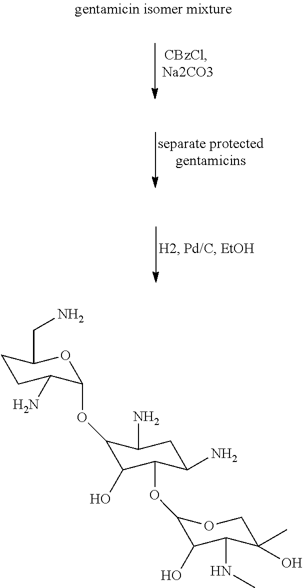

The different molecular forms were then isolated by HPLC as described below.

Separation method for the protected components using isocratic 70:30 acetonitrile:0.05% aqueous trifluoroacetic acid eluent

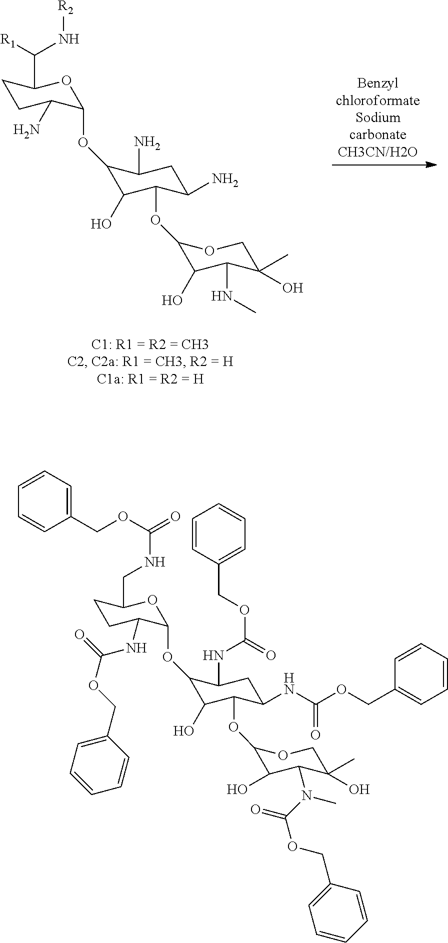

Crude protected gentamicin solution was prepared by dissolving protected gentamicin component mixture in anhydrous acetonitrile. The solution was analyzed as described on a YMC ODS AQ 4.6×150 mm column (1.0 mL/min) using an isocratic 70:30 acetonitrile:0.05% aqueous trifluoroacetic acid eluent with detection at 215 nm. The solution produced numerous peaks, including major peaks at 8.335 min (26.2% at 215 nm), 9.568 min (11.4% at 215 nm), 10.159 min (14.4% at 215 nmm), and 11.087 min (16.4% at 215 nm).

The separation between the first major peak and second major peak was only about 1.2 mins, and the second major peak and the third major peak were separated by less than a minute.

Conditions were sought whereby better separation could be achieved, allowing isolation of all components of CBz protected gentamicin.

Separation Method for the Protected Components Using Isocratic 65:35 Acetonitrile:0.05% Aqueous Trifluoroacetic Acid Eluent

A second analysis was completed as described above, except that an isocratic 65:35 acetonitrile:0.05% aqueous trifluoroacetic acid eluent was used. In this analysis, four major peaks eluted at 12.422 min (31.4% at 215 nm), 14.567 min (14.2% at 215 nm), 15.564 min (17.1% at 215 nm), and 17.141 min (19.6% at 215 nm).

Separation Method for the Protected Components Using Isocratic 63:37 Acetonitrile:0.05% Aqueous Trifluoroacetic Acid Eluent

A third analysis was then completed as described above, except that an isocratic 63:37 acetonitrile:0.05% aqueous trifluoroacetic acid eluent was used. In this final analysis, four major peaks were observed at 15.512 min (26.3% at 215 nm), 18.342 min (11.9% at 215 nm), 19.631 min (14.3% at 215 nm), and 21.7 min (16.4% at 215 nm). In this case, the first component is now separated from the second component by almost 3 mins, and the second and third components are now separated by 1.3 mins.

The above-described exemplary embodiments are intended to be illustrative in all respects, rather than restrictive, of the present invention. Thus, the present invention is capable of implementation in many variations and modifications that can be derived from the description herein by a person skilled in the art. All such variations and modifications are considered to be within the scope and spirit of the present invention as defined by the following claims.

What is claimed is:

1. A method of separating a gentamicin component, comprising the steps of protecting a gentamicin component mixture with a UV active protecting group, and purifying with HPLC.

2. The method of claim 1 further comprising deprotecting the gentamicin component.

3. The method of claim 2, wherein the deprotected gentamicin is not significantly degraded.

3. The method of claim 1 wherein the HPLC employs an acetonitrile eluent.

4. The method of claim 3 wherein the eluent is an isocratic acetonitrile aqueous trifluoroacetic acid eluent.

5. The method of claim 4 wherein the eluent has less than a 65:35 ratio of acetonitrile to 0.05% aqueous trifluoroacetic acid.

6. The method of claim 3 wherein the eluent is isocratic 63:37 acetonitrile:0.05% aqueous trifluoroacetic acid.

7. The method of claim 1, wherein the protecting group is selected from the group consisting of selected from a carbamate, an amide, an imide, benzyl, dimethoxyphenyl, dibenzosuberyl, trityl, picolyl N oxide, pyridyl N oxide, benzylidene, a benzylidene derivative, diphenylmethyl, a metal chelate, a phosphorus derivative, benzenesulfonyl, and a benzenesulfonyl derivative.

8. The method of claim 1, wherein the protecting group is a carbamate.

9. The method of claim 1, wherein the protecting group is a benzyl carbamate.

10. The method of claim 9, wherein the protecting group is benzyl carbamate substituted with chloro.

11. The method of claim 1, wherein the gentamicin component is gentamicin C1a.

12. A method of separating gentamicin C1a from a mixture of gentomicin components, comprising the steps of protecting a gentamicin component mixture with benzyl carbamate, and purifying with HPLC using an isocratic acetonitrile aqueous trifluoroacetic acid eluent wherein the eluent has less than a 65:35 ratio of acetonitrile to 0.05% aqueous trifluoroacetic acid.
